(19) United States
(12) Patent Application Publication
(10) Pub. No.: US 2002/0091796 A1
(43) Pub. Date: Jul. 11, 2002

(54) Method and apparatus for transmitting data over a network using a docking device

(76) Inventors: John Higginson, Santa Clara, CA (US); Deane Gardner, Cupertino, CA (US); Andreas Bibl, Los Altos, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/825,265

(22) Filed: Apr. 2, 2001

Related U.S. Application Data
(63) Continuation-in-part of application No. 09/476,142, filed on Jan. 3, 2000.

(51) Int. Cl. G06F 15/16; A61B 5/00; H04M 11/00; A63B 71/00

(52) U.S. Cl. 709/218; 709/203; 600/301; 128/904; 379/106.02; 482/8; 482/9

(57) ABSTRACT

A method and system for transmitting data over a network are described. In one embodiment, the system comprises a cradle to transmit data to a network server and an electronic device coupled to the cradle. The electronic device includes a sensor to trigger a data connection between the electronic device and the cradle. The electronic device also includes a transmitter to download the data to the cradle.

This application is a continuation-in-part of application Ser. No. 09/476,142, filed Jan. 3, 2000.

### BACKGROUND OF THE INVENTION

1. Field of the Invention

The present invention relates to data communication. More particularly, the present invention relates to transmitting data over a network using a docking device.

2. Background Information

Physical fitness is known to benefit people in many different areas, including improved flexibility and range of motion, increased muscular strength and cardiovascular fitness, body fat loss and increased stamina. Physical exercise helps maintain good health, increases energy, reduces stress and improves physical appearance. However, in order to gain the benefits of regular physical exercise, an exercise program needs to be carefully designed and correctly followed. It is desirable that a person engaged in a physical training program is guided by a professional fitness instructor or an athletic trainer. People with health problems may need to exercise under close supervision of a physician. A person who seeks to lose weight may need to get a recommendation on how to coordinate physical exercise and dieting.

In today's busy world, it is unlikely that many people can fully benefit from physical exercise unless the professional advice is readily available to them. However, the professional advice is only as good as the information provided to the professional by the person engaged in physical exercise. Conventional devices are known for obtaining this information via monitoring. Several prior art fitness monitoring devices are designed to provide the users with quick access to information concerning their exercise level. For example, U.S. Pat. No. 5,810,722 describes a device for measuring heartbeat rate. An athlete or a person engaged in fitness training may wear the device on the chest or the wrist. The device measures the heartbeat rate based on skin contact and allows the user to read the result from a display provided in the casing of the device. U.S. Pat. No. 5,891,042 describes a fitness monitoring device which includes an electronic pedometer integrated together with a wireless heart rate monitor. The device may be secured to the user's belt or waist band. The device receives electrical signals from a telemetric transmitter unit arranged on the user's skin adjacent to his heart and calculates the heart rate. The device is also configured to detect the user's body motion at each step for performing step counting. The user can read the results from a display provided in the casing of the device. The display includes an alpha/numeric display portion and a heart rate monitoring icon. These prior art devices, however, merely allow the users to see the physiological information concerning their exercise level. They do not provide any processed feedback or professional guidance to the users. In addition, these devices can be cumbersome to wear and they force the users to monitor their own activity, thereby interfering with their focus on physical exercise.

The prior art also includes fitness monitoring systems that allow some interaction between a user and a professional trainer. U.S. Pat. No. 5,598,849 describes a fitness monitoring system which includes a user monitor and a fitness system workstation. The monitor is mounted on a user's wrist and operates in conjunction with a heartbeat monitor mounted on a user's chest. The fitness system workstation is a local area network which includes a master computer and an interactive voice response computer. A personal trainer examines the user in a fitness center and obtains the physiological parameters for the user. Based upon these parameters, the master computer determines a suitable exercise regimen for the user. The personal trainer manually programs the user monitor and instructs the client in its use. During physical exercise, the user monitor indicates to the user whether his heart rate is above or below predetermined limits. The user can download data to the fitness system workstation by telephoning the fitness system workstation, holding the pulse code output against the telephone and actuate an appropriate push button on the keyboard to transfer data. At intervals, the fitness system workstation generates detailed reports relating to the user's performance. A personal trainer analyzes these reports and places a voice message for the user in the master computer. The voice message may be delivered to the user when the user telephones the fitness system workstation to download further exercise data.

Although the above prior art system provides some interactive monitoring of a user's exercise activity, this monitoring is very limited. In addition, the use of the system is cumbersome and requires visits to the fitness center. Furthermore, the feedback provided by the system is narrow and may not satisfy the user's needs with respect to physical exercise.

Therefore, what is required is an interactive fitness monitoring system which will make a wide variety of health and fitness information readily available to users and will effectively assist the users in their fitness activity.

### SUMMARY OF THE INVENTION

A method and system for transmitting data over a network are described. According to one aspect of the present invention, the system comprises a cradle to transmit data to a network server and an electronic device coupled to the cradle. The electronic device includes a sensor to trigger a data connection between the electronic device and the cradle. The electronic device also includes a data transmitter to download the data to the cradle.

### BRIEF DESCRIPTION OF THE DRAWINGS

The present invention is illustrated by way of example and may be better understood by referring to the following description in conjunction with the accompanying drawings, in which like references indicate similar elements and in which:

FIG. 1 is a block diagram of one embodiment for a network architecture;

FIG. 2 is a block diagram of one embodiment for an architecture of a computer system;

FIG. 3 is a flow diagram of one embodiment for managing data of subscribers via a network;

FIG. 4 is a block diagram of one embodiment for a personal data capture device;

FIG. 5 is a block diagram of another embodiment for a personal data capture device;

FIG. 6 is a flow diagram of one embodiment for monitoring data of subscribers using a personal data capture device;

FIG. 7 is a flow diagram of one embodiment for posting personal data of a subscriber on a web site;

FIGS. 8A-8C illustrate downloading personal data from a personal data capture device to a cradle; and

FIG. 9 is a flow diagram of one embodiment of a method for transmitting data over a network using a docking device.

### DETAILED DESCRIPTION OF AN EMBODIMENT OF THE PRESENT INVENTION

A method and system for transmitting data over a network using a docking device are described. In the following detailed description of the present invention, numerous specific details are set forth in order to provide a thorough understanding of the present invention. However, it will be apparent to one skilled in the art that the present invention may be practiced without these specific details. In some instances, well-known structures and devices are shown in block diagram form, rather than in detail, in order to avoid obscuring the present invention.

Some portions of the detailed descriptions that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of processing blocks leading to a desired result. The processing blocks are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention also relates to apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the invention as described herein.

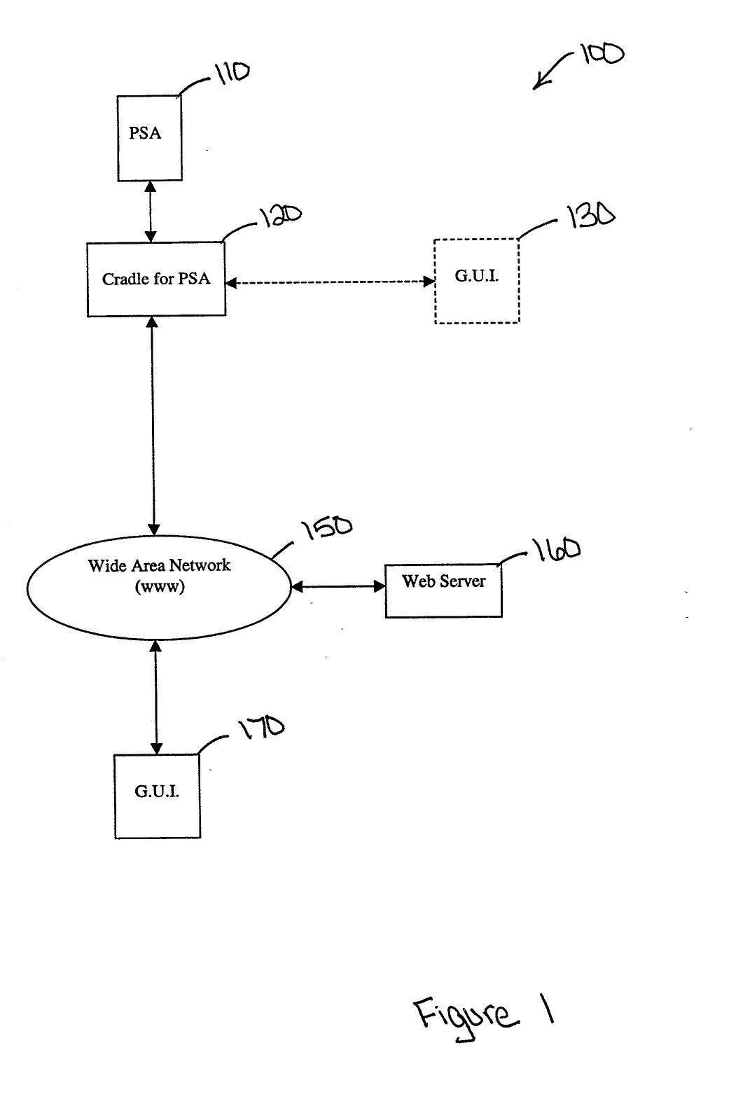

Referring now to FIG. 1, a block diagram of one embodiment for a network architecture is illustrated. In this embodiment, portable sports appliance (PSA) 110 is coupled to cradle 120. PSA 110 may be used to monitor and store physical and biometrical parameters of its user. In this embodiment, PSA 110 is a portable device. However, it will be recognized by one of ordinary skill in the art that a stationary device or a device included in some other device or equipment may be used with this invention without loss of generality. PSA 100 may be used by any person. For example, PSA 100 may be used by a person engaged in fitness activity, a professional athlete during exercise, or an employee wishing to know how his stress level changes during the day. The operation of PSA 110 and its functions will be described in more details below.

Cradle 120 is used to upload data from personal data capture device 100 to network 150. In one embodiment, cradle 120 may resemble a support element for a telephone receiver or handset. Cradle 120 may include a modem to transmit data over telephone lines and may be configured to provide two-way connection to wide area network 150.

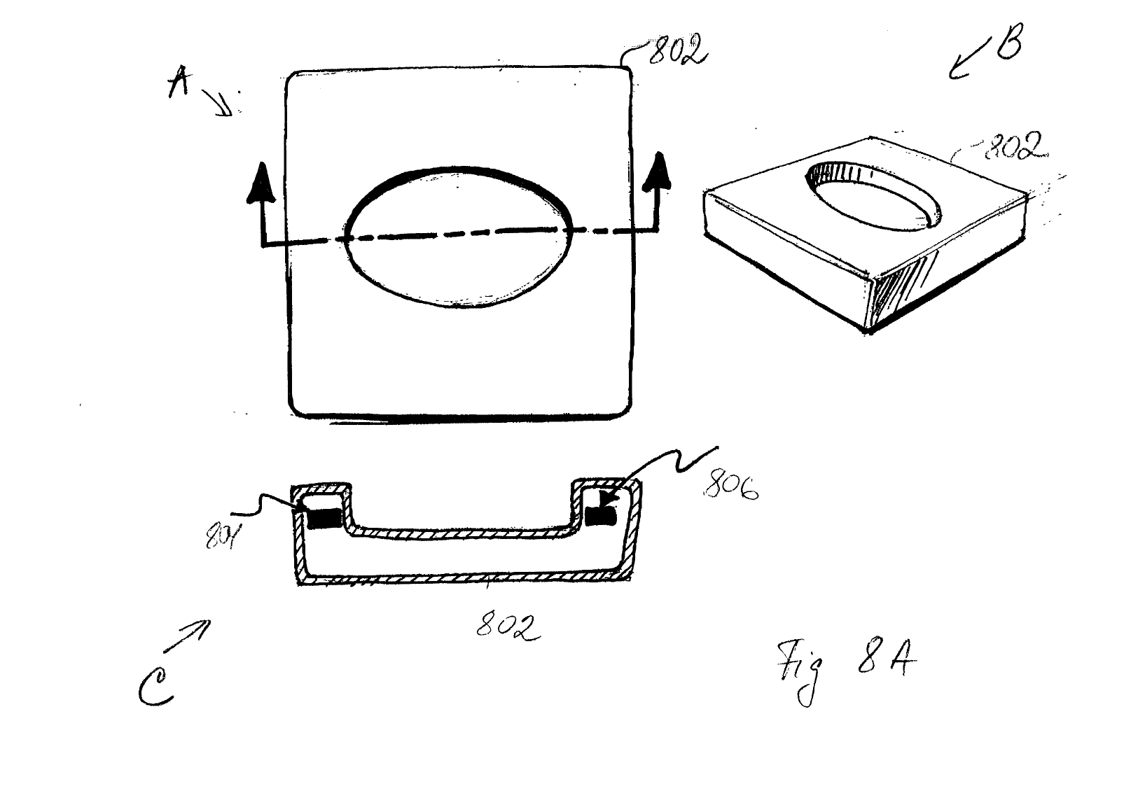

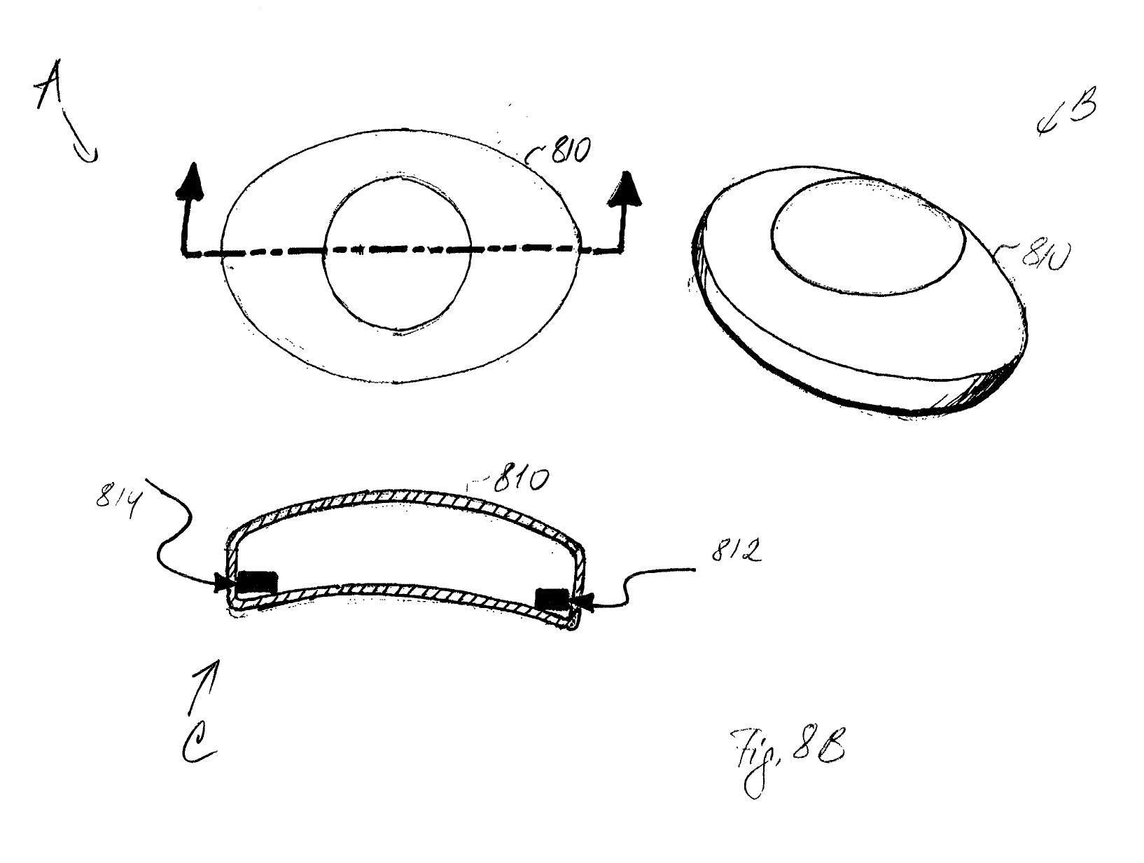

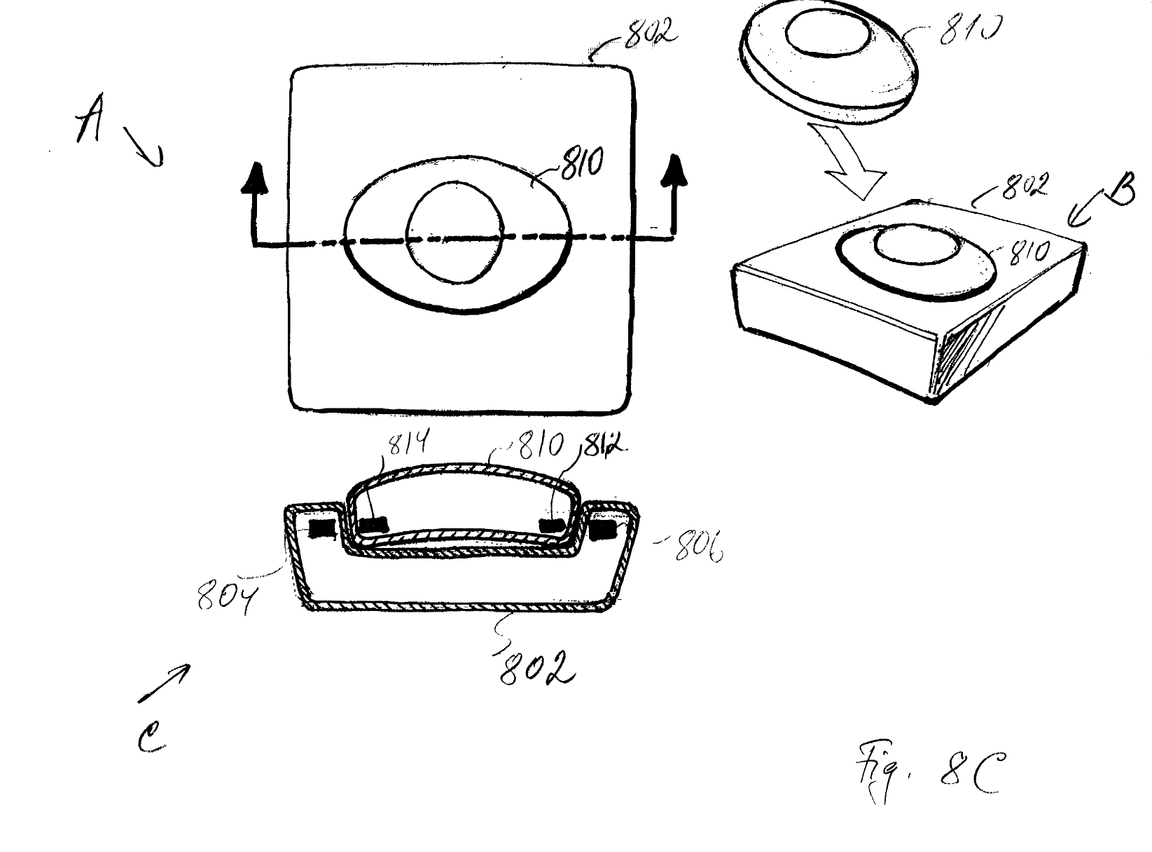

In one embodiment, placing personal data capture device 110 in cradle 120 may trigger an automatic dialing of a telephone number of server 160. When the telephone line is free, data from personal data capture device 110 may then be transmitted to server 160 via cradle 120 over wide area network 150. In one embodiment, the data is transmitted to cradle 120 via a two-directional infrared communication link between personal data capture device 110 and cradle 120. Alternatively, the data may be downloaded from personal data capture device 100 to cradle 120 using any data communication technique known in the art. One embodiment of downloading data to cradle 120 is described in greater detail below in conjunctions with FIGS. 8A-8C.

In another embodiment, the data may be transmitted from personal data capture device to server 160 using a wireless transmitter. That is, cradle 120 is not used, and the data is transmitted over a wireless carrier. It should be understood by one of ordinary skill in the art that various ways of transmitting data from PSA 110 to server 160, other than those described above, may be used with this invention without loss of generality.

Cradle 120 may be used with more than one PSA 110. For example, if each family member has his or her own PSA 100, cradle 120 may be shared by all family members. Personal data of each family member is then uploaded to server 160 at various points of time. Sever 160 may receive personal data from numerous PSA users. This personal data may then be processed by third parties that may provide feedback information to those PSA users who subscribe for this service.

In one embodiment, server 160 is coupled to wide area network 150. Wide area network 150 may include, for example, the Internet, America On-Line™, CompuServe™, Microsoft Network™, or Prodigy™. In addition, wide area network 150 may include, for example, conventional network backbones, long-haul telephone lines, Internet service providers, or various levels of network routers. Using conventional network protocols, server 160 may communicate through wide area network 150 to a plurality of clients.

In one embodiment, server communicates to clients 130 and 170. Clients 130 and 170 represent any device that may enable user's access to data. For simplicity, FIG. 1 shows only two clients, client 130 and client 170, that can communicate to server 160. However, it will be recognized by one of ordinary skill in the art that server 160 may communicate to a various number of clients and that a wide variety of client devices may be used with this invention without loss of generality. Such devices may include, for example, a conventional computer system, a network computer or thin client device (e.g., WebTV Networks™ Internet terminal or Oracle™ NC), a laptop or palm-top computing device (e.g., Palm Pilot™), a digital consumer device (e.g., a digital TV, a digital camcorder, or a “kitchen” computer”), etc. In one embodiment, clients 130 and 170 may have a Graphical User Interface (GUI) to allow users to access data. A GUI is a graphics-based user interface that incorporates icons, pull-down menus and a mouse. GUIs may include, for example, Microsoft Windows, Apple Macintosh, UNIX Motif, or UNIX OPENLOOK.

Clients 130 and 170 may be connected to server 160 in various ways. In one embodiment, clients 130 and 170 may be connected to server 160 through wide area network 150. Client 170 may represent client devices of third parties, e.g., health and fitness specialists, who access personal data of subscribers on server 160 via wide area network 150 to generate feedback information to subscribers. Client 130 may represent client devices of subscribers who access the generated feedback information via wide area network 150. In this embodiment, client 130 is connected to cradle 120 which provides two-way connection with wide area network 150. However, it will be understood by one of ordinary skill in the art that client 130 does not need to be connected to cradle 120. Instead, client 130 may use the same connection means as client 170.

In an alternate embodiment (not shown in FIG. 1), a client, such as client 130 or client 170, may be directly connected to server 160 or through a modem in a conventional way. When connected to wide area network 150, clients 130 and 170 may be connected directly to wide-area network 150 through direct or dial up telephone or other network transmission line. Alternatively, clients 130 and 170 may be connected to wide-area network 150 using a modem pool. A conventional modem pool may allow a plurality of clients to connect with a smaller set of modems in modem pool for connection to wide-area network 150. In yet another network typology, wide-area network 150 may be connected to a gateway computer, which may be used to route data to clients through a local area network. In this manner, clients can communicate with each other through a local area network (LAN) or with server 160 through a gateway and wide-area network 150. Alternatively, LAN may be directly connected to server 160 and clients may be connected through LAN. For example, subscribers' personal data may be processed by a company employing fitness instructors, athletic trainers, physicians and other heath and fitness specialists. Such a company may use LAN topology for providing internal communication between its employees. LAN may then be connected to server 160 through wide area network 150 for allowing communication between subscribers and health and fitness specialists.

Using one of a variety of network connection means, server computer 160 may communicate with clients 150 using conventional means. In one embodiment, a server computer 160 may operate as a web server if the World-Wide Web (WWW) portion of the Internet is used for wide area network 150. Using the HTTP protocol and the HTML coding language across a network, web server 160 may communicate across the World-Wide Web with clients 130 and 170. In this configuration, clients 130 and 170 may use a client application program known as a web browser such as the Netscape™ Navigator™ published by Netscape Corporation of Mountain View, Calif., the Internet Explorer™ published by Microsoft Corporation of Redmond, Wash., the user interface of America On-Line™, or the web browser or HTML translator of any other conventional supplier. Using such conventional browsers and the World-Wide Web, clients 130 and 170 may access graphical and textual data or video, audio, or tactile data provided by web server 160. Conventional means exist by which clients 130 and 170 may supply information to web server 160 through the World-Wide Web 150 and the web server 160 may return processed data to clients 130 and 170.

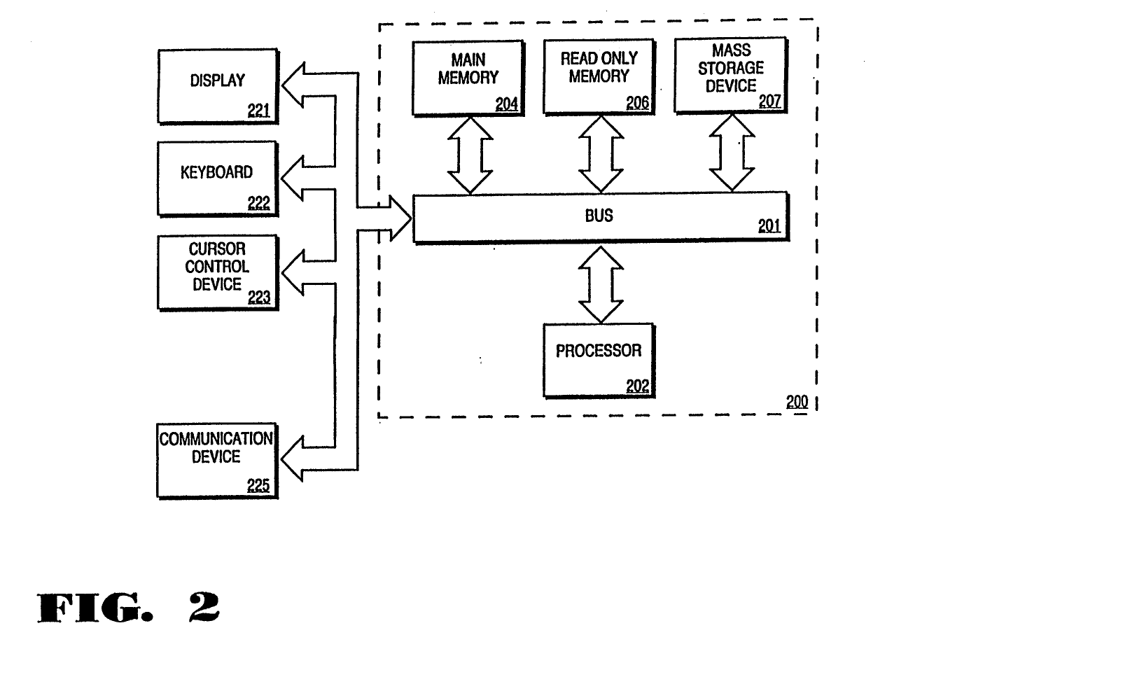

Having briefly described one embodiment of the network environment in which the present invention operates, FIG. 2 illustrates an example of a computer system 200 illustrating an exemplary client 130 or 170, or server 160 computer system in which the features of the present invention may be implemented. Referring to FIG. 2, computer system 200 is comprised of a bus or other communications means 201 for communicating information, and a processing means such as processor 202 coupled with bus 201 for processing information. Computer system 200 further comprises a random access memory (RAM) or other dynamic storage device 204 (commonly referred to as main memory), coupled to bus 201 for storing information and instructions to be executed by processor 202. Main memory 204 also may be used for storing temporary variables or other intermediate information during execution of instructions by processor 202. Computer system 200 also comprises a read only memory (ROM) and/or other static storage device 206 coupled to bus 201 for storing static information and instructions for processor 202.

An optional data storage device 207 such as a magnetic disk or optical disk and its corresponding drive may also be coupled to computer system 200 for storing information and instructions. Computer system 200 can also be coupled via bus 201 to a display device 221, such as a cathode ray tube (CRT) or a liquid crystal display (LCD), for displaying information to a computer user. For example, graphical or textual information may be presented to the user on display device 221. Typically, an alphanumeric input device 222, including alphanumeric and other keys is coupled to bus 201 for communicating information and/or command selections to processor 202. Another type of user input device is cursor control device 223, such as a conventional mouse, touch mouse, trackball, or other type of cursor direction keys for communicating direction information and command selection to processor 202 and for controlling cursor movement on display 221. A fully-loaded computer may optionally include video, camera, speakers, sound card, and many other conventional options.

Alternatively, clients 130 and 170 can be implemented as any device described above. Such a device does not necessarily include all of the elements and features of the above-described exemplary computer system; however, the functionality of the present invention may nevertheless be implemented with such devices.

A communication device 225 is also coupled to bus 201 for accessing remote computers or servers, such as web server 160, or other servers via the Internet, for example. The communication device 225 may include a modem, a network interface card, or other well known interface devices, such as those used for interfacing with Ethernet, Token-ring, or other types of networks. In any event, in this manner, the computer system 200 may be coupled to a number of servers 160 via a network infrastructure such as the infrastructure illustrated in FIG. 1 and described above.

FIGS. 8A-8C illustrate downloading data from an electronic device such as a personal data capture device 110 to a cradle, according to one embodiment of the present invention. Referring to FIG. 8A, an exemplary cradle 102 is shown that may be used to hold an electronic device (e.g., an electronic device 810 shown in FIG. 8B), to receive data from the electronic device, and to transfer this data to a network server such as web server 160. Cradle 802 includes a data receiver 804. Electronic device 810 includes a data transmitter 814 and a sensor 812. In one embodiment, an infrared connection is used to download data from electronic device 810 to cradle 802. In this embodiment, the data receiver 804 in cradle 802 represents an infrared receiver, and cradle 802 includes a magnet 806. Accordingly, the data transmitter 814 in the electronic device 810 represents an infrared transmitter and sensor 812 represents a magnetic read switch. It should be noted that cradle 802 and electronic device 810 may have various shapes and designs, and may include a variety of elements other that those described above.

In one embodiment illustrated in FIG. 8C, when electronic device 810 is placed into cradle 802, magnetic read switch 812 detects magnet 806 of cradle 802 and sends a signal to a microprocessor of electronic device 810. The microprocessor then activates infrared transmitter 814 which begins downloading data from electronic device 810 to cradle 802 using infrared receiver 804. When the downloading is completed, the infrared connection between electronic device 810 and cradle 802 is terminated. In one embodiment, the microprocessor of electronic device 810 terminates the infrared connection upon receiving a signal from infrared transmitter 814 indicating that the downloading has been completed. Alternatively, the microprocessor establishes the infrared connection for a predetermined period of time and terminates the infrared connection when this period of time expires.

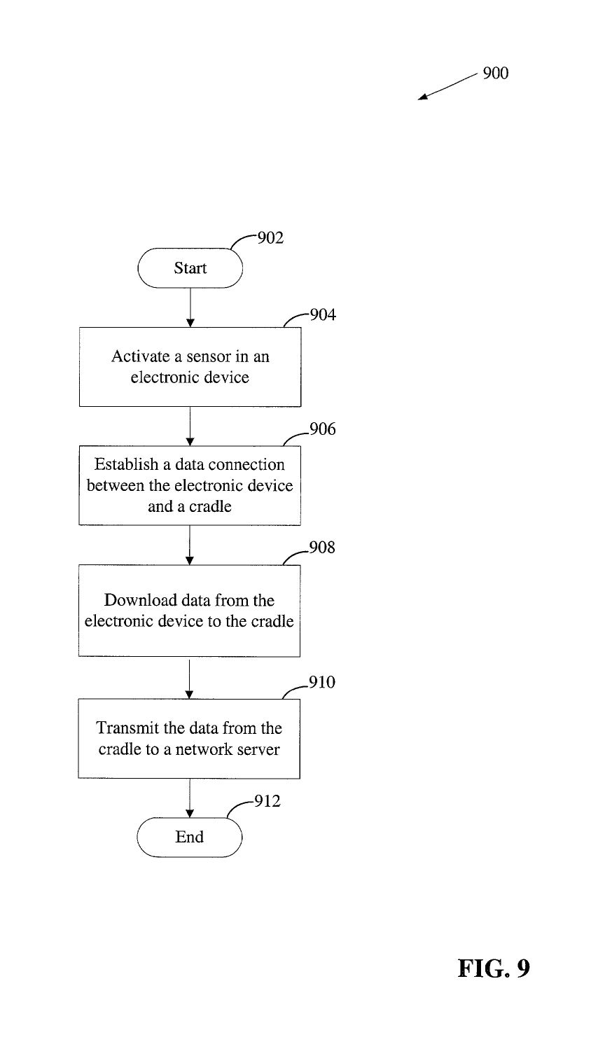

FIG. 9 is a flow diagram of a method 900 for transmitting data over a network using a docking device such as cradle 802. Method 900 begins with activating a sensor (e.g., magnetic read switch, a mechanical switch, etc.) in an electronic device such as a personal data capture device (processing block 904). In one embodiment, the sensor is activated when the electronic device is placed into the cradle. For example, a magnet or a mechanical means may be used to activate the sensor.

In response to the activation of the sensor, a data connection is established between the electronic device and the cradle (processing block 906). In one embodiment, the data connection is infrared.

At processing block 908, data captured in the electronic device is downloaded to the cradle. In one embodiment, the electronic device captures personal data of the user of the electronic device. The user may also be a subscriber of a service provided by a network server as will be described in greater detail below.

When the downloading is completed, the data connection is terminated. In one embodiment, the connection is terminated when a data transmitter of the electronic device sends a signal indicating that the downloading has completed. In an alternative embodiment, the data connection is terminated when a predefined download time period has expired. In either embodiment, the data connection is established only for a limited period of time to minimize power consumption of the electronic device and cradle.

Afterwards, at processing block 910, the data is transmitted from the cradle to a network server. In some embodiments, the data is transmitted using a modem. In other embodiments, the data may transmitted from the cradle to the network server via a wireless carrier or using any other data communication technique known in the art.

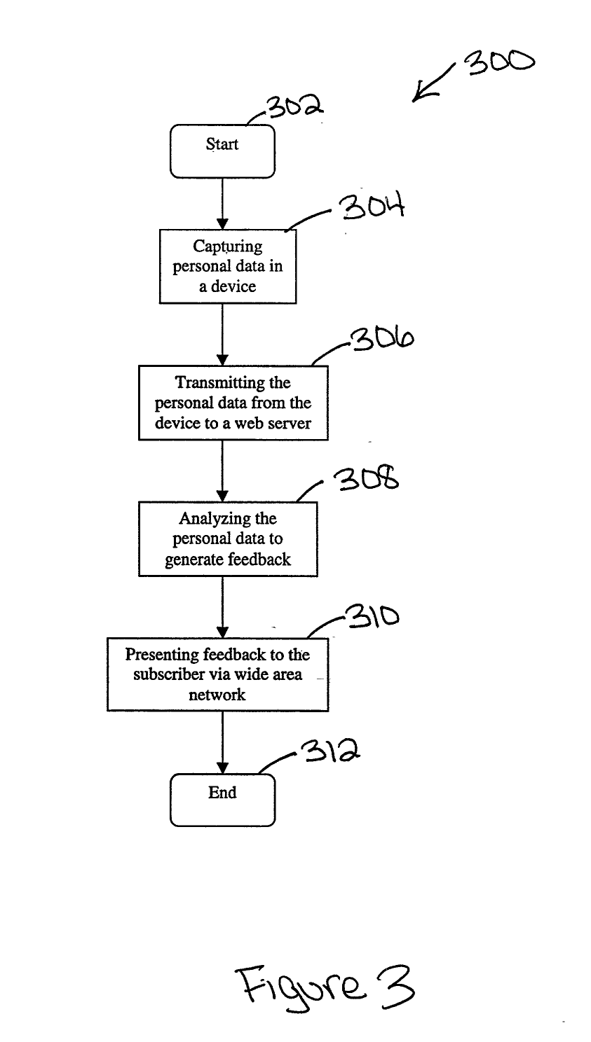

FIG. 3 is a flow diagram of one embodiment for managing data of subscribers via a network. At processing block 304, personal data of a subscriber is captured in a personal data capture device. The personal data includes physical and biometrical parameters of the subscriber. These parameters may be measured at any time and during any activity of the subscriber, including, for example, physical exercise, work related activities, or quiet time at home. The process of measuring the parameters and capturing them in the personal data capture device will be described in more detail below.

At processing block 304, the personal data is transmitted from the personal data capture device to a network server. In one embodiment, the personal data may be transmitted using a cradle. That is, the personal data capture device is placed in the cradle which triggers an automatic dialing of a telephone number of the server. When the telephone line is free, data from the personal data capture device may be transmitted to the web server through a wide area network. The transmitted data may include a unique identifier associated with the data capture device. Alternatively, the cradle may not be used, and the personal data may be transmitted to the web server using a wireless transmitter via a wireless carrier.

The server stores personal data of subscribers. In one embodiment, each subscriber is required to have an account registered at the web server in order to receive services provided by the web server. The account may need to be created before personal data is first transmitted to the web server. In alternate embodiments, the account may be created when the personal data capture device is purchased or at the time of first transmission of personal data to the web server.

At processing block 308, the personal data is analyzed to generate feedback information. In one embodiment, the personal data on the web server may be accessed by a third party. The third party may include, for example, a fitness instructor, an athletic trainer, a diet or nutrition specialist, a physician, or any other fitness or health specialist. One or more specialists may have access to the personal data of the subscriber depending on the subscriber's needs and subscribed services. Every specialist involved in the subscribed services may create feedback information based on the personal data of the subscriber.

At processing block 310, the feedback information is presented to the subscriber over a wide area network. In one embodiment, the subscriber accesses the feedback information upon entering a password. The password may be associated with the subscriber's account. Alternatively, the subscriber may need to enter a code associated with the personal data capture device or any other unique information allowing to prevent access to the subscriber's personal data by outsiders. It will be understood by one of ordinary skill in the art that various other ways of maintaining confidentiality of the subscriber's personal data may be used with the present invention without loss of generality.

In one embodiment, the feedback information is posted on a private web site of the subscriber. Alternatively, the subscriber may access the feedback information on a particular web site known to all subscribers (e.g.,/www.sportbrain.com/) upon entering a password or a certain unique code. In addition to the feedback information, the personal data may be presented to the subscriber in numerous forms. The numerous forms may include, for example, various graphs, tables, map overlays, progressive charts, and comparisons with data of other subscribers.

In one embodiment, the personal data capture device may be configured from the web site by the subscriber. Alternatively, the personal data capture device may be configured by a health or fitness specialist over the wide area network. For example, a fitness instructor may decide to reconfigure the personal data capture device according to a new version of the exercise program that the instructor designed for the subscriber based on the recent personal data of the subscriber. In yet another embodiment, both the subscriber and the instructor may have the ability to reconfigure the personal data capture device over the network.

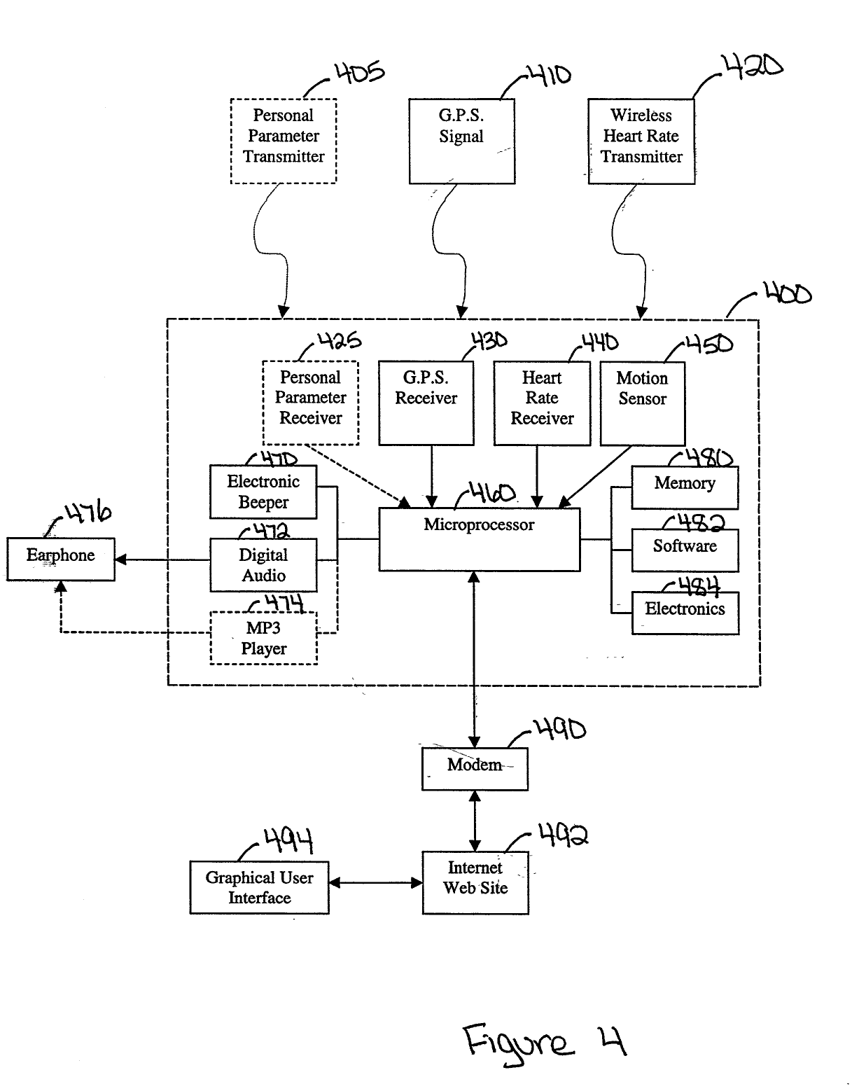

The personal data capture device will now be described in more detail. FIG. 4 is a block diagram of one embodiment for a personal data capture device. Referring to FIG. 4, personal data capture device 400 includes microprocessor 460 which is coupled to memory 480, software program 482 and electronics 484. In one embodiment, personal data capture device 400 is a portable device. In this embodiment, personal data capture device 400 may be clipped to the user's waist band, or may be secured to the user in other ways such as via a wrist watch-type arrangement or by simply being held by the user. In alternate embodiments, personal data capture device 400 may be a stationary device or a device included in some other device or equipment.

In one embodiment, personal data capture device 400 includes a global positioning system (GPS) signal receiver 430 which receives GPS signals 410. GPS signals 410 may include three-dimensional positional information and velocity of the user when the user is walking or running, or is engaged in some other relevant activity. Personal data capture device 400 may also include a motion sensor 450 which may improve the accuracy of the above information or substitute it when GPS signal 410 is interrupted. In addition, personal data capture device 400 may include heart rate receiver 430 which receives heart beat rate from wireless heart rate transmitter 420. Various other sources may provide signals to personal data capture device 400. Personal parameter transmitter 405 represents a wide variety of signals that may be received by various personal parameter receivers 425 included in personal data capture device 400. For example, a bathroom weight scale may have a transmitter built in it to automatically transmit signals with weight data to personal data capture device 400. A blood pressure meter, a glucose meter, exercise equipment such as treadmills and stationary bikes, or any other device or equipment can transmit data to personal data capturing device 400 which will receive it using a corresponding personal parameter receiver 425. In one embodiment, heart rate receiver 440 is personal parameter receiver 425. It will be understood by one of ordinary skill in the art that all of the receivers described above or any combination of them may be included in personal data capture device 400 without loss of generality.

Upon receiving a signal, any of the receivers 425 through 450 outputs data to microprocessor 460. Microprocessor 460 stores this data in memory 480. In one embodiment, personal data capture device may include electronic beeper 470 for providing audio signals related to the personal data. For example, when personal data capture device 400 is configured over the network as described above, electronic beeper 470 can be set to signal low and high heart rate target limits, low and high pace limits, low and high weight limits, etc. Alternatively, or in addition to electronic beeper 470, personal data capture device 400 may include digital audio 472 to provide more detailed feedback. Digital audio 472 may be configured to provide various real time information (e.g., distance average speed) related to personal parameters transmitted to personal data capture device 400. Personal data capture device 400 may also include digital audio player such as MP3 player 474 to play digital recordings. In one embodiment, the real time information generated by digital audio 472 and/or digital recordings played by MP3 player may be communicated to the user by earphone 476 coupled to digital audio 472 and/or MP3 player 474. It will be understood by one of ordinary skill in the art that any combination of personal data receivers 425 through 450 and audio signal generators 470 through 476 may be used with personal data capture device 400 without loss of generality.

In one embodiment, microprocessor 460 is coupled to modem 490 which is contained in a cradle. Modem 490 provides a direct two-way connection to web server 160 and subscriber's web site 492 over a wide area network. In this embodiment, when personal data capture device 400 is placed in the cradle, modem 490 automatically dials a phone number of web server 160. When the phone line is free, software 482 causes microprocessor 460 to upload subscriber's personal data from memory 480 to web server 160. Alternatively, personal data capture device may include a wireless transmitter (not shown) which may be used by microprocessor 460 to transmit the subscriber's personal data from memory 480 to web server 160 via a wireless carrier.

In one embodiment, the subscriber's personal data may be posted on subscriber's web site 492. Alternatively, web site 492 may be a company web site which can be accessed by all subscribers. GUI 494 is coupled to web site 492 to provide the user with a convenient way to view the data.

In one embodiment, the user may be provided with an option of adding new features to personal data capture device 400 over the wide area network. For example, the user may be notified of availability of a new or improved version for personal data capture device 400. The user may then be allowed to download the new or improved version directly over the Internet.

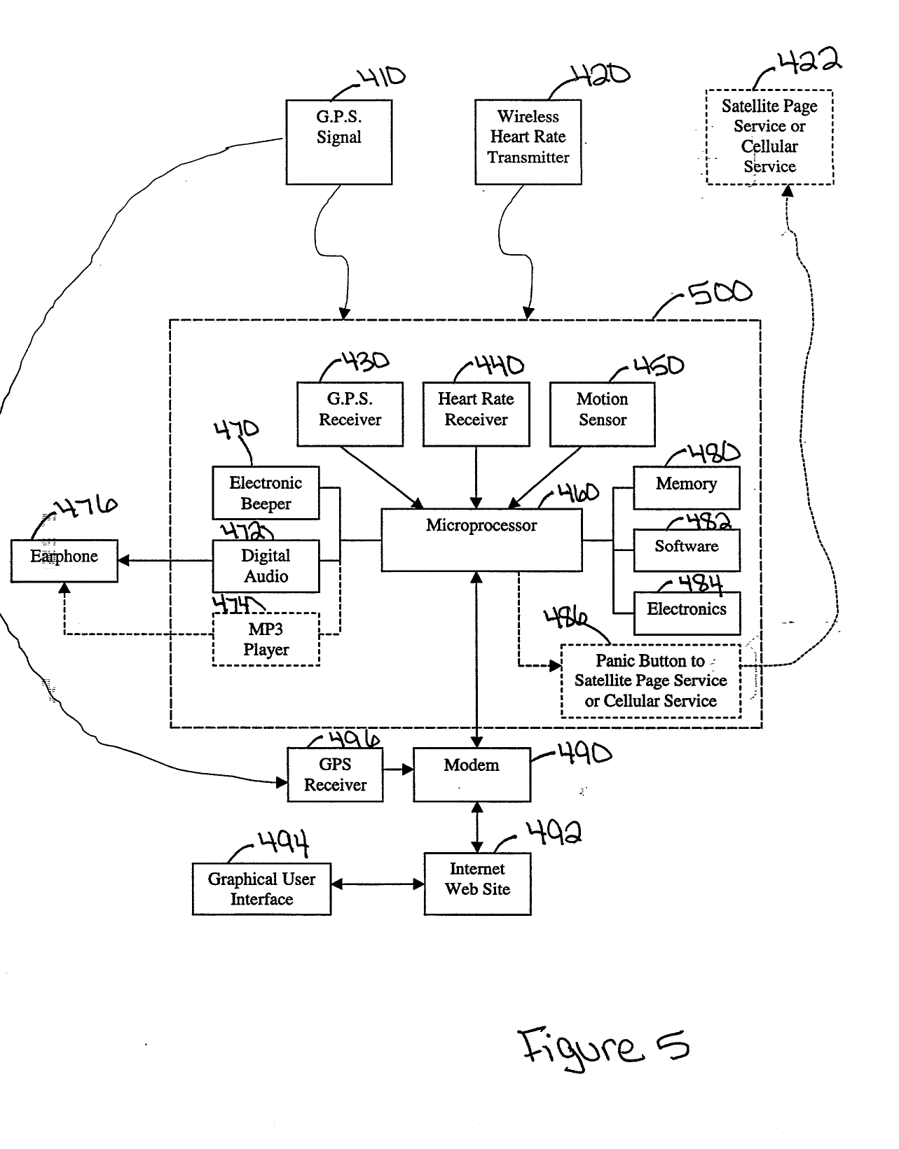

FIG. 5 is a block diagram of another embodiment for a personal data capture device. Referring to FIG. 5, personal data capture device 500 may include the same features and functionality as personal data capture device 400. In one embodiment, personal data capture device 500 includes all data receivers 425 through 450 and all audio signal generators 470 through 476. In alternate embodiments, personal data capture device 500 may include any combination of receivers 425-450 and audio signal generators 470-476.

In one embodiment, panic button 486 is coupled to microprocessor 460. In this embodiment, software 482 analyzes personal data in memory 480. If the personal data includes a parameter that is below or exceeds a certain panic parameter (e.g., heart rate is too low or too high), software 482 may cause microprocessor 460 to invoke panic button 486. Panic button 486 may then produce a panic signal to a satellite page service or a cellular service 422. In one embodiment, the panic parameters may be set or updated from the user's web site 492 and transferred to memory 480 over the wide area network. In alternate embodiments, the panic parameters may be set or updated by health or fitness specialists in web server 160 or programmed during the manufacture of personal data capture device 500.

In one embodiment, GPS receiver 496 is coupled to modem 490. GPS receiver 496 receives signals directly from GPS 410 to provide differential correction of GPS signals. Alternatively, differential correction of GPS signals may be done from a personal web site of a subscriber or from a company web site.

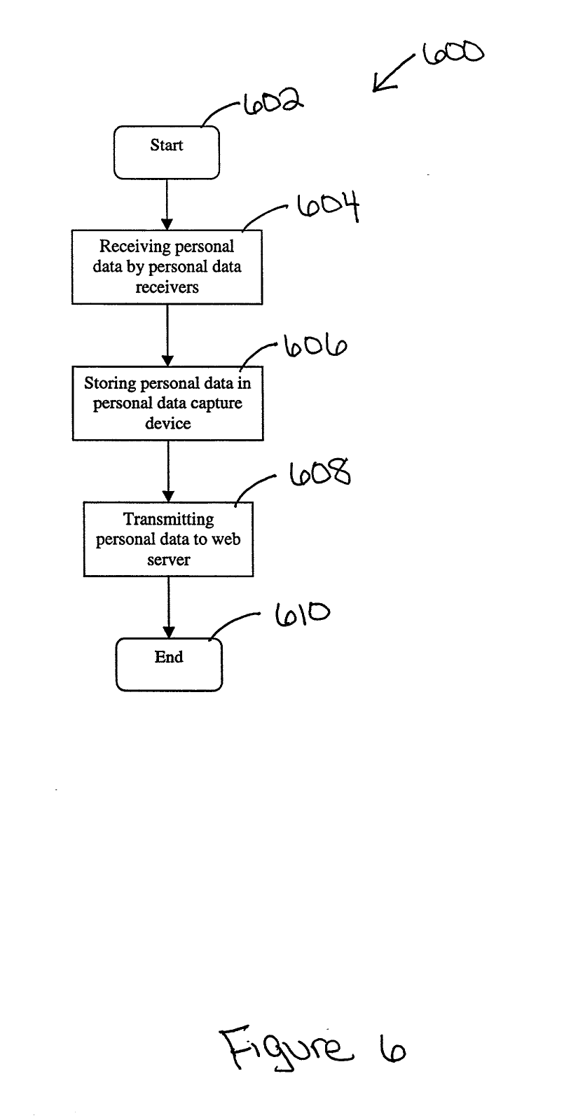

FIG. 6 is a flow diagram of one embodiment for monitoring data of subscribers using a personal data capture device. Referring to FIG. 6, at processing block 604, personal data is received by personal parameter receivers. As described above, the personal data may be received from one or more sources. Personal parameter receivers may include, for example, a GPS signal receiver, a heart rate receiver, a motion sensor, a weight data receiver, a blood pressure receiver, a glucose measurement receiver, or an exercise data receiver.

At processing block 606, the personal data is stored in the personal data capture device. The personal parameter receivers output the personal data to microprocessor 460 which stores the personal data in memory 480 of the personal data capture device. The personal data may include a timestamp and information identifying a source of a personal parameter. In one embodiment, when the personal data includes certain parameters, an audio signal may be produced by electronic beeper 470. In addition, certain real time information may be communicated to the user by digital audio 472 and earphone 476 based upon the personal data. Alternatively or in addition to the above audio signals, when the personal data includes a panic parameter, a panic signal may be generated to a satellite page service or a cellular service.

At processing block 608, the personal data is transmitted from memory 480 to the web server via the wide area network. In one embodiment, the personal data may be transmitted over a phone line using cradle 120 which may have a direct two-way connection to the Internet. Alternatively, the personal data may be transmitted from the personal data capture device to the web server using a wireless transmitter via a wireless carrier. The personal data may then be posted on the subscriber's web site.

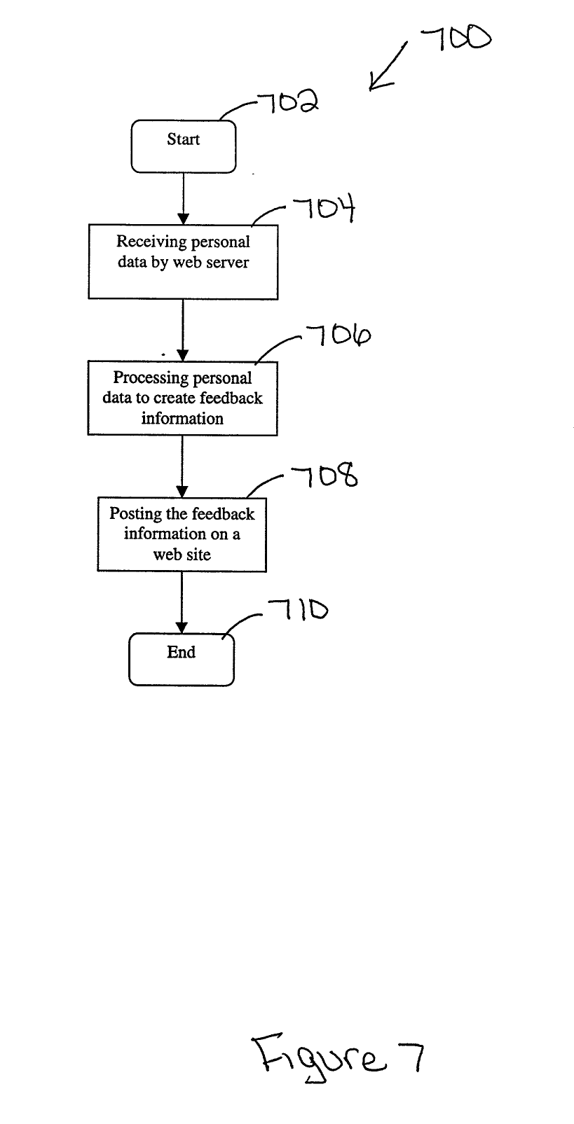

FIG. 7 is a flow diagram of one embodiment for posting personal data of a subscriber on a web site. Referring to FIG. 7, at processing block 704, web server 160 receives personal data of subscribers. In one embodiment, the personal data may be received from a plurality of personal data capture devices. The personal data may comprise physical data and biometrical parameters of each subscriber. The personal data may be stored in a repository of personal data which resides either directly on web server 160 or on a separate computer accessible by web server 160.

In one embodiment, the personal data of the subscriber is stored in the repository of personal data only if the subscriber maintains an account registered with web server 160. The account may be registered at any time before or simultaneously with first transmission of subscriber's personal data. At the time of creating the account, the subscriber may be required to provide a password or a code to prevent access to the personal data by anyone other than the subscriber. In one embodiment, the account information may be stored together with the personal data in the repository of the personal data. Alternatively, the account information may be stored in a separate database or file.

At processing block 706, the personal data is processed to create feedback information. Depending on the services selected by the subscriber, various feedback information may be created in response to the subscriber's personal data. In one embodiment, a fitness instructor, an athletic trainer, a diet or nutrition specialist, a physician, or any other fitness or health specialist may be able to access the subscriber's personal data. One or more fitness or health specialists may analyze the personal data and create the feedback information. In alternate embodiments, the personal data or its portion may be analyzed by a software program which may either create the feedback information entirely or assist fitness or health specialists in creating the feedback information. The feedback information may be stored either in the repository of personal data or in a separate database residing on web server 160 or on a different computer accessible by web server 160.

At processing block 708, the feedback information is posted on a web site. As described above, the web site may be a personal web site of the subscriber or a company web site that can be accessed by all subscribers. In one embodiment, the personal data may be posted on the web site in various forms such as graphs, tables and map overlays. In addition, the subscriber's personal data may be compared with personal data of other subscribers or with this subscriber's history data. In one embodiment, when the personal web site is used, the web site may be specifically created as a part of services provided to the subscriber. Alternatively, the subscriber's existing web site may be used for posting the feedback information and the personal data of the subscriber. In yet another embodiment, the feedback information and personal data may be posted on a company web site known to all subscribers. In either embodiment, access to the feedback information and personal data is protected either by a password or other means for maintaining confidentiality of personal information.

Several variations in the implementation of the method and system for transmitting data over a network using a docking device have been described. The specific arrangements and methods described here are illustrative of the principles of this invention. Numerous modifications in form and detail may be made by those skilled in the art without departing from the true spirit and scope of the invention. Although this invention has been shown in relation to a particular embodiment, it should not be considered so limited. Rather it is limited only by the appended claims.

What is claimed is:

1. A method of transmitting data over a network, the method comprising:
activating a sensor in an electronic device;
in response to the activation of the sensor, establishing a data connection between the electronic device and a cradle;
downloading data from the electronic device to the cradle; and
transmitting the data from the cradle to a network server.

2. The method of claim 1 wherein the data connection is an infrared connection and the sensor is a magnetic read switch.

3. The method of claim 1 wherein the sensor is activated when the electronic device is placed into the cradle.

4. The method of claim 1 further comprising terminating the data connection between the electronic device and the cradle when the downloading is completed.

5. The method of claim 1 wherein the data is personal data of a user of the electronic device.

6. The method of claim 1 further comprising presenting the data to a user on a web site.

7. The method of claim 6 wherein the web site is a personal web site of the user or a company web site accessible by the user.

8. The method of claim 1 wherein the electronic device is a personal data capture device.

9. The method of claim 1 wherein the data is transmitted to the network server using a wireless transmitter via a wireless carrier.

10. The method of claim 1 wherein the data is transmitted to the network server using a modem via a wide area network.

11. A system comprising:
a cradle to transmit data to a network server; and
an electronic device, coupled to the cradle, the electronic device including
a sensor to trigger a data connection between the electronic device and the cradle, and
a transmitter to download the data to the cradle.

12 The system of claim 1 wherein the data connection is an infrared connection and the sensor is a magnetic read switch.

13. The system of claim 11 wherein the sensor is activated when the electronic device is placed into the cradle.

14. The system of claim 11 wherein the electronic device includes a processor to terminate the connection between the electronic device and the cradle when the download is completed.

15. The system of claim 11 wherein the data is personal data of a user of the electronic device.

16. The system of claim 11 further comprising a web site to present the data to a user.

17. The system of claim 16 wherein the web site is a personal web site of the user or a company web site accessible by the user.

18. The system of claim 11 wherein the electronic device is a personal data capture device.

19. The system of claim 11 wherein the cradle includes a receiver to receive the data from the electronic device.

20. The system of claim 19 wherein the receiver is an infrared receiver.

21. The system of claim 11 wherein the cradle includes a wireless transmitter to transmit the data to the network server via a wireless carrier.

22. The system of claim 11 wherein the cradle includes a modem to transmit the data to the network server via a wide area network.

23. A computer readable medium that provides instructions, which when executed on a processor, cause said processor to perform operations comprising:
activating a sensor in an electronic device;
in response to the activation of the sensor, establishing a data connection between the electronic device and a cradle;
downloading data from the electronic device to the cradle; and
transmitting the data from the cradle to a network server.
